(19) United States
(12) Patent Application Publication
(10) Pub. No.: US 2006/0207596 A1
(43) Pub. Date: Sep. 21, 2006

(54) Device and method for delivery of combination nasal medication

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials, LLC, Bridgeport, CT (US)

(21) Appl. No.: 11/239,348

(22) Filed: Sep. 30, 2005

Related U.S. Application Data
(60) Provisional application No. 60/662,800, filed on Mar. 18, 2005.

(51) Int. Cl. A62B 7/10; A61M 15/08

(52) U.S. Cl. 128/206.11; 128/207.18

(57) ABSTRACT

The present invention relates to dual nasal spray applicator devices and systems for administration of two nasal compositions in one spray or aerosol dose without mixing of the compositions prior to administration and methods of treatment using these devices and systems. The devices allow incompatible drug or other compositions to be stored and administered by a single device in a single dose, remaining separated until just prior to or during administration of the medications.

This application is a nonprovisional of prior co-pending provisional application U.S. Ser. No. 60/662,800, filed March 18, 2005 and claims the benefit thereof. U.S. Ser. No. 60/662,800 is hereby incorporated by reference in its entirety.

### BACKGROUND OF THE INVENTION

1. Field of the Invention

The present invention relates generally to medical devices for administration of pharmaceutical compositions. In particular, this invention relates to a dual nasal applicator system for simultaneous nasal administration of two liquid compositions by spray, micronization or aerosolation, to the nasal passages of a patient, simultaneously in a single dosage. Methods and treatments using the application device also form part of the invention.

2. Description of the Background Art

Nasally administered medications are known in the art, for topical administration to the nasal mucosa. As used in this application, the terms “nasal drug,” “nasal medication” and “nasal composition” refer to any pharmaceutical composition (such as, for example a drug, a saline solution, a vaccine or insulin) which may be administered nasally, and includes liquid or semi-liquid formulations (e.g. solutions, gels or suspensions) that contain the pharmaceutical composition. Examples of commonly used nasal medications and other compositions for nasal administration include, but are not limited to, antihistamines, decongestants, corticosteroids, cromolyn, hot pepper compositions, and homeopathic or herbal compositions.

While most patients with nasal symptoms or need for a nasally administered medication use only one therapeutic agent at a time, a significant number either do not respond adequately to a single agent or require treatment for more than one symptom or set of symptoms. Such patients may require a combination of drugs to provide the desired relief. For example, mucosal inflammation and swelling caused by the body's response to the presence of the allergen(s) in the nasal sinuses can prevent topical medications from reaching the affected area or reaching the affected area in adequate amounts or concentrations.

Combining a nasal steroid with a second composition, such as a topically active antihistamine, reduces inflammation to allow better penetration of the second composition into the nasal passages. The result, in the case of the combination of a nasal steroid and antihistamine is an antihistaminic action in the sinuses with an improved therapeutic effect over antihistamine alone.

However, not all drugs are compatible in a single liquid formulation suitable for nasal administration. Such drugs either can only be formulated in a dual combination with extreme difficulty or cannot be formulated in a dual combination at all, due to chemical instability or widely differing chemical or physical properties that preclude dissolution in the same solvent. For these combinations, it has been necessary to provide two separate nasal sprays with two separate dispensers to administer the compositions separately.

It is well-known that development of new formulations is a time- and labor-intensive process, as well as involving considerable expense. Therefore, even if two drugs are compatible such that they can be prepared in a fixed combination medication, such as a single solution or suspension containing the two drugs, it still would be desirable to provide a mechanism whereby the two drugs can be administered simultaneously without having to develop and test a new single formulation.

Medical therapy for seasonal allergic rhinitis (SAR) and perennial allergic rhinitis (PAR) traditionally has relied on oral antihistamine medications for treatment of symptoms and nasal decongestants to alleviate congestion in the sinuses. Recently, however, nasal spray antihistamine formulations and intranasal corticosteroids have become available. Of course, nasal decongestants are known for nasal administration.

SAR and PAR both are common reasons for application of a nasal medication or more than one nasal medication. Formulations for treatment of allergic rhinitis that incorporate both an intra-nasally delivered topical antihistamine and an intra-nasally delivered steroid in a single composition have not been available commercially. The combination of medications in a single nasally delivered pharmaceutical preparation would provide improved simplicity in dosing, improved patient compliance, and significant cost savings to both the patient and the patient's insurance carrier by avoiding the necessity of obtaining and administering two separate pharmaceutical compositions. Unfortunately, there has been considerable difficulty in formulating such a combination nasal spray product with an antihistamine drug compound and a nasal steroid compound because of their incompatability. A combination antihistamine/corticosteroid product for nasal administration is not available currently.

Antihistamine drugs such as acrivastine, azatadine, azelatine, brompheniramine, carbmoxamine, chlorpheniramine, citirizine, clemastine, cyproheptadine, desloratadine, dexbrompheniramine, dexchlorpheniramine, diphenhydramine, doxylamine, fexofenadine, hydroxyzine, levocabastine, loratadine, meclizine, methdilazine, methscopolamine, phenindamine, pheniramine, phenylpropanolamine, promethazine, pyrilamine, rocastine, trimeprazine, tripelennamine, and triprolidine, for example, are known for treatment of allergies and nasal allergies in particular. Some of these drugs have been formulated for oral administration or as nasal sprays, eye drops, or other topical medications. Those that have been formulated are water-soluble and have been formulated, whether for a nasal spray or nose drop formulation, in the form of an aqueous solution.

Nasal steroid drugs, on the other hand, such as beclomethasone, budesonide, dexamethasone, flunisolide, fluticasone, mometasone, and triamcinolone, are virtually insoluble in water and only sparingly soluble in ethanol. These drugs cannot be formulated as an aqueous solution. Therefore, when a spray formulation for the nasal passages is desired, steroid drugs usually are formulated as a suspension of microfine particles of drug in an aqueous carrier.

Formulating a combination medication containing both a nasal steroid in suspension and an antihistamine in solution, with an aqueous carrier suitable for application to the nasal mucosa, has not been possible. Reliable drug activity and dose, as well as stability, have not been achievable using methods known in the art.

Decongestant medications such as oxymetazoline, phenylephrine and tetrahydrolazine are known in the art for nasal application. The drug cromolyn also has wide application for nasal symptoms and has been administered as a spray for nasal allergies. Other compositions known for administration to the nasal passages include hot pepper sprays and various homeopathic and herbal remedies. In addition, certain vaccines may be administered nasally. Of interest recently is the proposed formulation of dual antidiabetic medications for nasal administration, such as insulin and a traditional antihyperglycemic drug.

A conventional aerosol nasal dispenser device includes a head incorporating a substance-dispensing orifice, at least a portion of the head being designed to be inserted into the nostril of the user for the purpose of dispensing a medication, and a body for accommodating the medication. The user actuates the dispenser device to expel a dose, preferably a measured dose, of micronized or aerosolized medication into the nasal passages.

Generally, the body of known nasal medication dispenser devices contain a medication or a mixture of medications in a liquid vehicle, optionally containing additional excipients such as preservatives, buffers, diluents, solvents, suspending agents, perfumes, flavorings and the like. There is a need in the art, however for a nasal dispenser device and method that can deliver two separate drugs, such as, for example, an antihistamine and a nasal steroid, in a single application such that the two drugs are not mixed together until just prior to dispensing or upon aerosolation.

### SUMMARY OF THE INVENTION

A general object of the present invention is to overcome the aforementioned problems of administration of nasally administered drug compositions and to improve patient compliance with a particular medication regime, thereby improving patient healing and comfort by enabling administration of two drugs in a single dose. One embodiment of the present invention provides a dual chambered applicator for intranasal delivery of at least two different medications which are maintained in separate chambers and conduits until aerosolized or otherwise mixed for dispensing into the nasal passages of a user.

Accordingly, in one embodiment, the present invention provides a dual nasal applicator system for simultaneously delivering a first nasal drug composition and a second nasal drug composition housed separately in first and second composition chambers, respectively, comprising a spray head device comprising a nasal insertion portion having a body member and first and second openings extending through said body member, and an actuation portion that is integral with said nasal insertion portion for delivering the first and second nasal drug compositions through said first and second openings, respectively, when actuated; a first connecting collar device having a first lower connecting portion and a first upper connecting portion, said first lower connecting portion being sealably attached to the first composition chamber and said first upper connecting portion being attached to said spray head device; a second connecting collar device having a second lower connecting portion and a second upper connecting portion, said second lower connecting portion being sealably attached to the second composition chamber and said second upper connecting portion being attached to said spray head device; a dual dispenser system having a first dispenser device and a second dispenser device; said first dispenser device comprises a first lower portion member extending through and beneath said first connecting collar device to form a first liquid conduit from the first composition chamber, and a first upper portion member communicable with said first lower portion member for delivering said first nasal drug composition housed in the first composition chamber to said first opening of said spray head; and said second dispenser device comprises a second lower portion member extending through and beneath said second connecting collar device to form a second liquid conduit from the second composition chamber, and a second upper portion member communicable with said second lower portion member for delivering said second nasal drug composition housed in the second composition chamber to said second opening of said spray head, wherein the first and second nasal drug compositions remain separated until dispensed into a user's nares. In preferred systems, the spray head device has at least one of an oblong shape, circular shape, square shape, rectangular shape, and octagonal shape, and is configured in different widths and heights.

In preferred systems, the first nasal composition and said second nasal composition are dispensed as an aerosol, and may be dispensed in dispensed in equal volumes or dispensed in unequal volumes. Preferably, the compositions are dispensed in metered volumes.

Preferred embodiments of the present invention comprise an antihistamine drug composition and a nasal steroid drug composition. The antihistamine drug composition may comprise loratadine, desloratidine, terfenidine, fexofenadine, cetirizine, azelatine, azatadine, clemastine, brompheniramine, chlorpheniramine, dexbrompheniramine, diphenhydramine, doxylamine, phenindamine, pheniramine, pyrilamine, triprolidine, levocabastine, acrivastine, carbmoxamine, dexchlorpheniramine, promethazine, trimeprazine, methdilazine, hydroxyzine, rocastine, tripelennamine, meclizine, tripolidine, cyproheptadine, methscopolamine or phenylpropanolamine. Preferred embodiments of the present invention also comprise a nasal steroid drug composition. The nasal steroid drug may comprise fluticasone, dexamethasone, flunisolide, budesonide, triamcinolone, beclomethasone or beclomethasone.

Preferably, the antihistamine drug composition is formulated in an aqueous solution and further comprises a buffer for maintaining the aqueous solution at pH 6.5-7.2 and/or a preservative agent. Preferably, the nasal steroid drug composition comprises a microfine nasal steroid drug in aqueous suspension and further comprises a preservative agent.

Additional first or second nasal compositions may be selected from the group consisting of an antihistamine, a decongestant, a corticosteroid, cromolyn, a hot pepper composition, a homeopathic remedy, an herbal composition, a vaccine, a saline solution a sexual dysfunction compounds, a pain management compound, a cytokine, a migraine medication, a hormone, an osteoporosis medication, insulin, an antihyperglycemia medication, a vitamin, a mineral, a nutraceutical, a sedative, a motion sickness medication, a Parkinson's disease medication, an immunogen, a medication for stroke protection, a gene therapy compound, an otitis media treatment compound, a treatment for cognition and neuronal survival, a medications for real or suspected narcotic overdose, an asthma medication, an anticonvulsant, an antiemetic, an infertility treatment compound, a growth factor, an antiviral drug, an antimicrobial drug, a cardiac drug, a vaccine and an adjuvant.

In further embodiments the present invention provides a method of treating nasal allergies in a patient in need thereof which comprises nasally administering to the patient an antihistamine drug composition and a nasal steroid drug composition with a dual nasal applicator system as described above.

### BRIEF DESCRIPTION OF THE DRAWINGS

The accompanying drawings, which are incorporated herein and form part of the specification, illustrate various embodiments of the present invention. These drawings, together with the description, further explain the principles of the invention and enable a person skilled in the pertinent art to make and use the invention. In the drawings, like reference numbers indicate identical or functionally similar elements.

FIG. 1 is a drawing of the exterior of an exemplary embodiment of the dual nasal applicator of the present invention.

FIG. 2 is a drawing which shows a top view of a preferred exemplary embodiment of the nasal spray head of the present invention.

FIG. 3 is a drawing showing a side view of the exterior of an exemplary embodiment of the dual nasal applicator of the present invention.

FIG. 4 is a drawing showing a side view of the exterior of an exemplary embodiment of the dual nasal applicator of the present invention at a 90° vantage point from the view of FIG. 3.

FIG. 5 is a longitudinal cross-sectional view of a preferred embodiment of the dual nasal applicator taken at line B-B of FIG. 4.

FIG. 6 is a drawing illustrating an exterior view of a preferred embodiment of the nasal spray head of the present invention.

FIG. 7 is a drawing showing a side view of the embodiment illustrated in FIG. 6.

FIG. 8 is a cross-sectional view of a preferred embodiment of the nasal spray head taken at line A-A of FIG. 7.

FIG. 9 is a side view of a preferred embodiment of the nasal spray head of the present invention.

FIG. 10 is a longitudinal cross-sectional view of a preferred embodiment of the nasal spray head taken at line G-G of FIG. 9.

FIG. 11 is an exterior view of a preferred embodiment of the connecting collar of the present invention.

FIG. 12 is a side view of the connecting collar of FIG. 11.

FIG. 13 is a longitudinal cross-sectional view of the connecting collar taken at line C-C of FIG. 12.

FIG. 14 is a drawing of an exterior view of a preferred embodiment of the delivery conduit of the present invention.

FIG. 15 is a side view of the delivery conduit of FIG. 14.

FIG. 16 is a longitudinal cross-sectional view of the delivery conduit taken at line F-F of FIG. 15.

FIG. 17 is an exterior view of the pump body of a preferred embodiment of the present invention.

FIG. 18 is an exterior view of the piston of a preferred embodiment of the present invention.

FIG. 19 is a side view of the piston of FIG. 18.

FIG. 20 is a longitudinal cross-sectional view of the piston taken at line K-K of FIG. 19.

FIG. 21 is a drawing of an exterior view of a preferred embodiment of the crossfeed of the present invention.

FIG. 22 is a bottom view of the crossfeed of FIG. 21.

FIG. 23 is a longitudinal cross-sectional view of the crossfeed taken at line L-L of FIG. 22.

FIG. 24 is a drawing of an exterior view of a preferred embodiment of the insert of the present invention.

FIG. 25 is a side view of the insert of FIG. 24.

FIG. 26 is a side view of the insert of FIG. 24 at a 90° vantage point from the view illustrated in FIG. 25.

FIG. 27 is a longitudinal cross-sectional view of the insert taken at line M-M of FIG. 25.

### DETAILED DESCRIPTION OF THE PREFERRED EMBODIMENTS

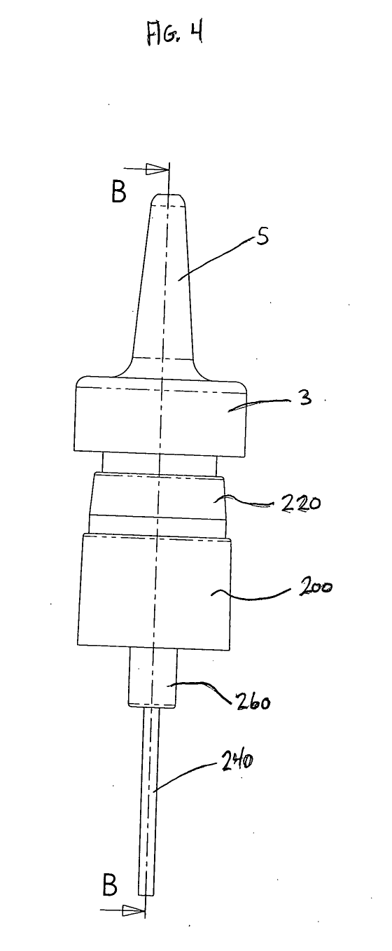

FIG. 1 illustrates an exterior view of the dual nasal applicator 1. The dual nasal applicator 1 of the present invention dispenses separately-housed first and second nasal compositions to the nasal passages of a user in one dose or application. The two nasal compositions are separate until immediately prior to or during administration of the compositions to the nasal passages of the user. Referring to FIG. 1, the dual nasal applicator 1 comprises two connecting collars 12, for sealed attachment of two nasal composition containers (not shown). The dual nasal applicator 1 also comprises a dual dispenser system 10 (FIG. 5). Each of two dispenser systems are situated centrally inside each connecting collar 12. Each dispenser system 10 has a lower portion that extends through and beneath the connecting collar 12 to form a liquid conduit 253-257 (FIG. 5) from the nasal composition container.

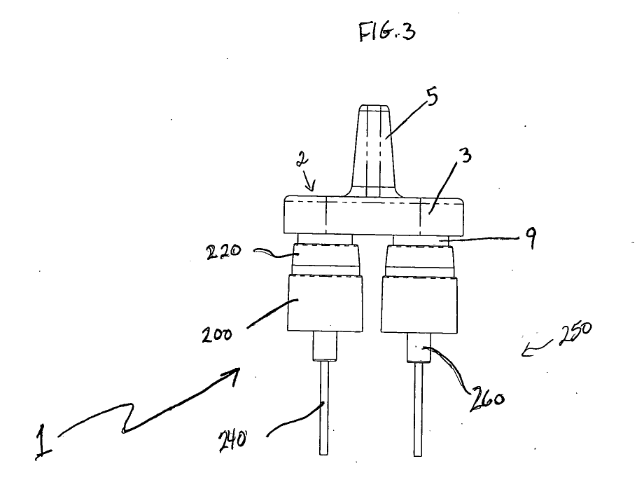

Each connecting collar 12 has an upper connecting means 220, having an interior and an exterior, that surrounds a flange 9 (FIG. 3) without physically contacting the same. The connecting collar 12 contains in its interior a means for attachment 451 (FIG. 5), such as an annular bayonet connection means, to the upper portion 270 of delivery means 250. The preferred means of connection between the upper connecting means 220 and the delivery means is a bayonet-type attachment. However, the person of skill can easily recognize any number of suitable connecting means that provide a method for removably or non-removably attaching the upper portion 260 of the delivery means to the interior of the upper connecting means 220, for example, friction connectors, screw connectors, or the like.

The upper connecting means 220 is located above the lower connecting means 200. Together, the upper 220 and lower 200 connecting means form the connecting collar 12, which serves to physically attach the liquid nasal composition container in a liquid transferable manner with the nasal spray head 2, such that the nasal compositions each may be dispensed through the separate openings 7, 8 in the nasal spray head 2. The dispenser system 10 thus is disposed beneath and sealably adjacent to the nasal spray head 2.

The nasal spray head 2 comprises an actuation region 3 and a nasal insertion member 5. The nasal spray head 2 sits on the dispenser systems 10 by means of the flange 9 both to facilitate comfortable insertion of the device into the nares and provide a means for actuation to deliver the nasal compositions through the dual openings 7, 8 in the nasal insertion member 5.

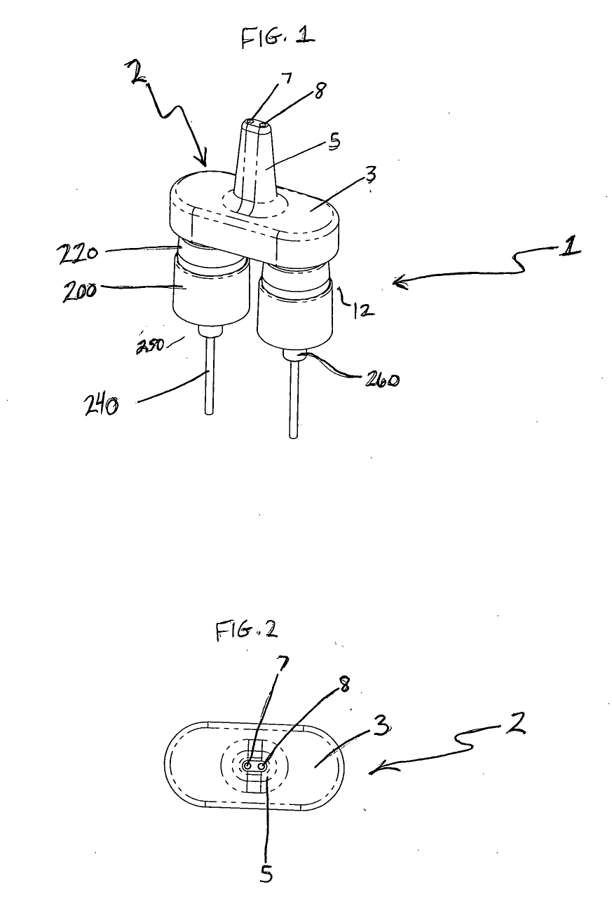

Referring to FIG. 2, which provides a top view of the nasal spray head 2, the nasal spray head 2, in this embodiment, has a body having a lower portion which forms an actuation region 3 which is open at the lower end and a protruding upper portion which forms the nasal insertion member 5. It will be appreciated by those skilled in the art that the nasal spray head 2, although described and shown in the drawings to have a generally oblong shape, also can have any cross-sectional shape such as, for example, circular, square, rectangular, octagonal, or the like, and can be configured in different widths and heights as is convenient. Therefore, different general shapes and configurations are contemplated in addition to oblong, so long as the functions of the nasal spray head described herein are maintained. Likewise, the relative height of the nasal insertion member 5 and the width of the actuation region 3 of the nasal spray head 2 may have any convenient dimension(s).

FIG. 3 is a side view of one embodiment of the dual nasal applicator 1. Referring to FIG. 3, the nasal spray head 2 is situated above and is optionally removably attached to the dispenser system 10. The dispenser system 10 comprises two tubular liquid conduit and delivery means 250, each of which is disposed longitudinally at the center of the lower connecting means 200 and extends below the lower connecting means 200 for insertion into an individual nasal composition container of appropriate dimensions.

The tubular liquid conduit and delivery means 250 also are known as “delivery conduits.” Each comprises an upper end and a lower end, and are shown in the drawings as having a cylindrical shape with a circular cross section. The tubular liquid conduit and delivery means 250, however, encompass conformations of delivery conduits with a circular, square, rectangular, octagonal or any other cross-sectional shapes, and are referred to and depicted in the drawings as cylindrical only for convenience. The lower end of each delivery conduit 250 is open to admit liquid nasal composition into the delivery conduit for transfer of the nasal composition. In preferred embodiments, each delivery conduit 250 is disposed longitudinally at the center of the nasal composition container which is attached at the center of the lower connecting means 200, shown for convenience in the drawings as a screw-type connector.

FIG. 4 shows a side view of the dual nasal applicator 1 at a 90° vantage point from the side view of FIG. 3. Thus, only one dispenser system 10 is visible, including the upper connecting means 220 that extends through and above the lower connecting means 200 and the delivery conduit 250.

FIG. 5 is an illustration showing a longitudinal cross-sectional view of a preferred embodiment of the nasal applicator 1 taken at line B-B of FIG. 4. Referring to FIG. 5, each delivery conduit is a hollow, preferably cylindrical, conduit, having a liquid transferring interior 253 and an opening 251, 252 at the lower end for admission of fluid from the nasal composition container. For the sake of convenience, only one dispenser system 10 of the dual nasal applicator 1 will be described in detail below. However, it will be understood by the skilled person that the dual nasal applicator 1 comprises at least two dispenser systems 10 as is shown in FIGS. 3 and 5. Each dispenser system 10 is a mirror image of the other, contains the same components and serves the same function, e.g., each dispenser system 10 provides a means to deliver liquid nasal composition from one nasal composition container for delivery through the corresponding opening 7 or 8 of the nasal insertion member 5 into the nares of a user.

The upper end of the delivery conduit 250 widens to form a wider central cylindrical zone 260 for accommodation of a spring 300, and widens a second time to form an upper cylindrical zone 270 which contains a connection means 450 at is upper extremity for attachment to the connecting collar 12. The spring 300 has a lower end and an upper end. The lower end of the central cylindrical zone 260 forms a perpendicular annular base 262 on which the lower end of the spring 300 rests. The upper end of the cylindrical zone 260 houses both the spring 300 and a pump body 310.

The pump body 310 is a solid cylindrical body having an annular ridge 312 which rests on the upper end of the spring 300, allowing force from the spring to be transmitted to piston 320. The piston 320 is a hollow, preferably cylindrical, body that defines a liquid accommodating channel having upper and lower ends, and has an interior side and an exterior side. The lower end of the piston 320 rests in the interior of the upper end of the delivery conduit 250 and protrudes from said upper end, forming a liquid transferring interior continuation 255 of the liquid transferring interior 253 of the delivery conduit 250. Thus, the lower end of the piston 320 fits movably but snugly inside the upper cylindrical zone 270 of the delivery conduit 250. The piston 320 is held firmly in the upper cylindrical zone 270 of the delivery conduit 250.

The opening at the lower end of the liquid transferring continuation 255 of the piston 320 rests on the upper end of the pump body 310 and is movable by compression of spring 300.

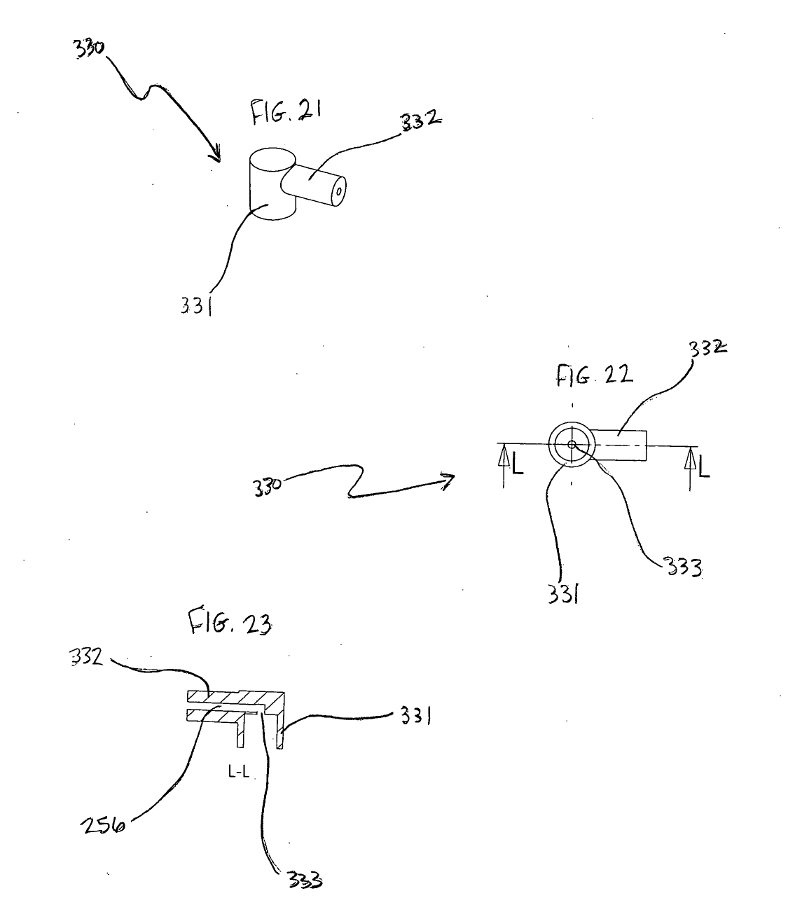

The opening at the upper end of the liquid transferring continuation 255 of the piston 320 meets flush with an opening 333 (FIG. 22) at the lower end 331 of a crossfeed 330. The crossfeed 330 fits in a liquid sealing manner over the upper end of the piston 320, such that liquid moves from the liquid transferring continuation 255 to a liquid transferring conduit 256 (FIG. 23).

As illustrated in FIGS. 21 through 23, the crossfeed 330 is an L-shaped hollow body with an upper end 332 and a lower end 331, each of which has an opening, that forms a further liquid transferring conduit 256, allowing liquid to be transferred further upward from the upper end of the piston 320 and transversely or centrally. The upper end 332 of the crossfeed 330 fits in a liquid sealing manner into the lower end of a single insert 340 (FIG. 24) such that the liquid transferring conduit 256 is flush with the liquid transferring conduit 257 of the insert 340, allowing transfer of liquid from the liquid transferring conduit 256 to the liquid transferring conduit 257.

Referring to FIG. 24, the insert 340 is a partially hollow L-shaped body with a solid upper end and with a lower end that accommodates the liquid transferring conduit 257. The lower end is designed to accept the upper end of the piston 320 in a liquid sealing manner and has an opening which joins with the opening in the lower end of the crossfeed 330 to form a further liquid transferring conduit 257 by which liquid may be transferred from the crossfeed 330. The lower end of the insert 340 forms a conduit for the nasal composition from the opening at the terminus of the lower end and upward.

Referring to FIGS. 25 through 27, the liquid transferring conduit 257 of the insert 340 traverses the lower end of the insert 340 and is continuous with an exit port 288 located on the side of the insert 340. Liquid nasal compositions traveling from the nasal composition container through the delivery conduit 250 and the different portions of the liquid transferring conduit 253-257 exit at exit port 288. It will be seen by those of skill in the art that the exact shape and manner of attachment of the piston 320, crossfeed 330 and insert 340 is not essential to the invention so long as these parts fit together to form a sealed liquid conduit to carry the liquid nasal compositions to the exit port 288. The upper portion of the insert 340, which is the portion of the insert located above the exit port 288, is a solid, cylindrical body. The upper cylindrical body and a portion of the lower portion of the insert 340 fits inside the nasal insertion member 5 of the nasal spray head 2, as described in detail below.

Referring back to FIG. 5, the nasal insertion member 5 is a generally cylindrical or truncated cone-shaped portion of the nasal spray head 2, which preferably is oblong or elliptical in cross-section to accommodate two openings 7, 8 for administration of liquid nasal compositions into the nasal passages of a user. In the interior of the nasal insertion member 5, contiguous in a liquid transferring-capable manner with each opening 7, 8 in the nasal insertion member 5, are two cylindrical chambers 298 having a lower open end and an upper opening 7, 8. The cylindrical chambers 298 each accommodate the upper solid portion of the insert 340 and a sufficient portion of the insert 340, such that the exit port 288 communicates with the cylindrical chamber in which the insert lies.

The diameter of the upper portion of the insert 340 is sufficiently narrow to allow the insert to fit within the cylindrical chamber 298 leaving an annular space surrounding the insert 340. However, below the exit port 288, at least a portion of the insert 340 is of a greater diameter. Thus, the insertion of the insert 340 into the cylindrical chamber 298 creates a liquid seal at or near the lower open end of the cylindrical chamber 298 that prevents flow of liquid from the exit port 288 out the lower end of the cylindrical chamber 298.

The length of the upper portion of the insert 340 is sufficient to fill the entire length of the cylindrical chamber 298 up to the opening at its upper end, resulting in an annular opening 7, 8 when the insert 340 is fully inserted into the cylindrical chamber 298 forming a seal at its lower end, as shown in FIG. 5. Alternatively, the length may be shorter than the full length of the cylindrical chamber 298 so that the opening 7, 8 is circular. As will be appreciated by any person of skill, any configuration is suitable so long as movement of the liquid nasal compositions through the annular passageway and out of the openings 7, 8 results in a spray or aerosol administration of the liquid nasal compositions when the nasal insertion member 5 is placed in the nares of a user and activated.

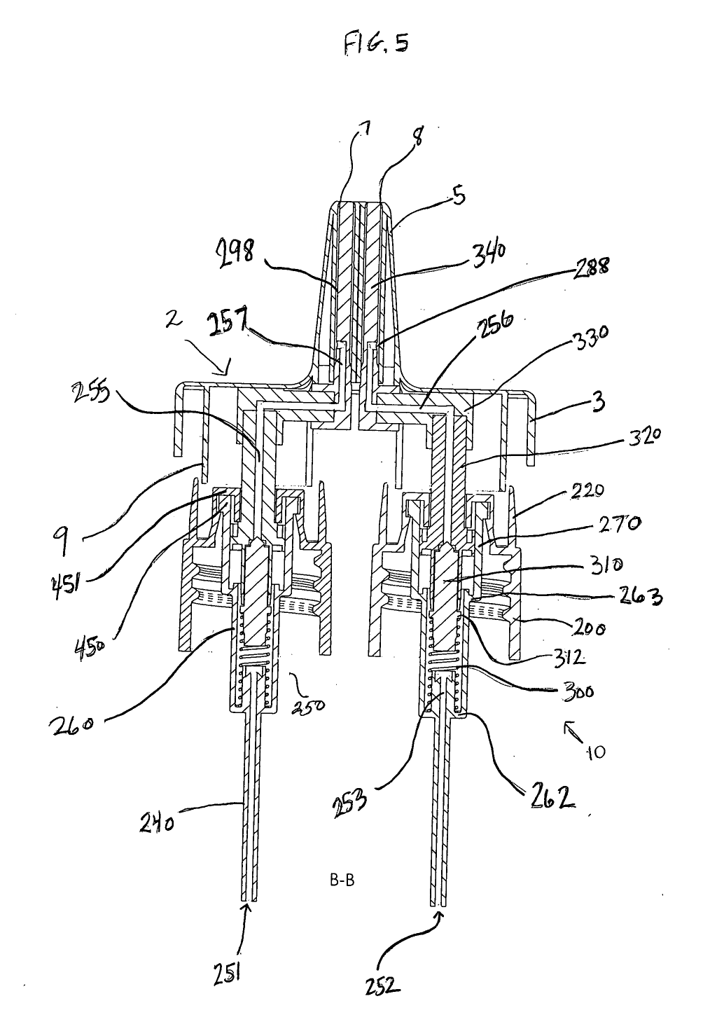

FIG. 6 is an illustration providing an exterior view of the nasal spray head 2. Referring to FIG. 6, the nasal spray head 2 comprises an actuation region 3 with flanges 9 for accommodation of the upper connecting means 220 of the dispenser system 10 connecting collar 12 and through which the piston 320 is positioned (see FIG. 5). The nasal insertion member 5 of the nasal spray head 2 contains dual openings 7, 8 through which the liquid nasal compositions are dispensed. FIG. 7 is a side view of the nasal spray head 2 which illustrates the nasal insertion member 5, actuation means 3 and flanges 9 of the nasal spray head 2.

FIG. 8 is a cross-sectional view of the nasal spray head 2 taken at line A-A of FIG. 7 through the nasal insertion member 5 of the nasal spray head 2. The cylindrical chamber 298 of the nasal spray head 2 is shown with the insert 340 placed inside the cylindrical chamber 298.

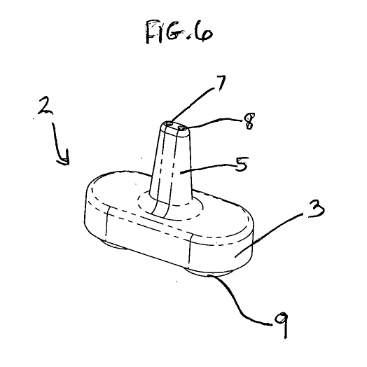

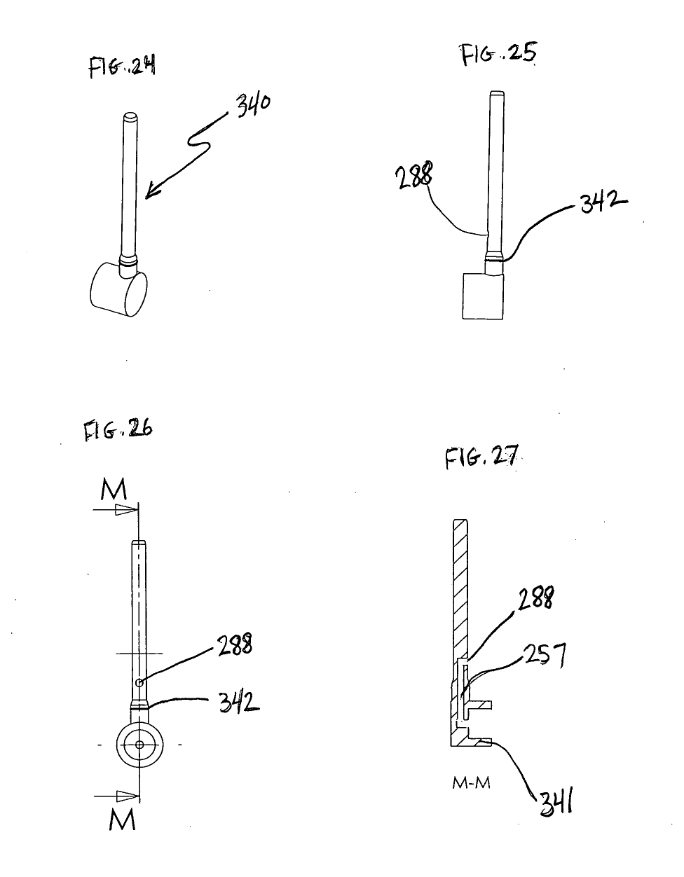

FIG. 9 is a side view of the nasal spray head 2 showing the actuation means 3, flanges 9 and nasal insertion member 5. Referring to FIG. 10, which illustrates a longitudinal cross-section of a preferred nasal spray head 2 taken at line G-G of FIG. 9, the cylindrical chamber 298 is shown having an optional and preferred constriction of the diameter of the chamber at the upper end where the opening 7, 8 is located. Also depicted is an optional sealing means 299 near the lower open end of the cylindrical chamber 298 which acts to seal the chamber 298 from liquid flow out of the chamber 298 in a downward direction once the insert 340 is in place. Optionally, the inside wall of the cylindrical chamber 298 may contain an annular groove to accommodate a seal, for example, an O-ring. Alternatively, the insert 340 may contain a seal means 342 (FIGS. 25 and 26), such as an annular groove to accommodate a seal, such as an O-ring.

FIG. 11 illustrates a preferred embodiment of the connecting collar 12, which comprises an upper connecting means 220 and a lower connecting means 200. In this view, the upper aspect of the annular bayonet connection means 451 of the preferred embodiment is depicted. The upper connecting means 220 is annular in configuration, with a center opening 455 through which the piston 320 passes.

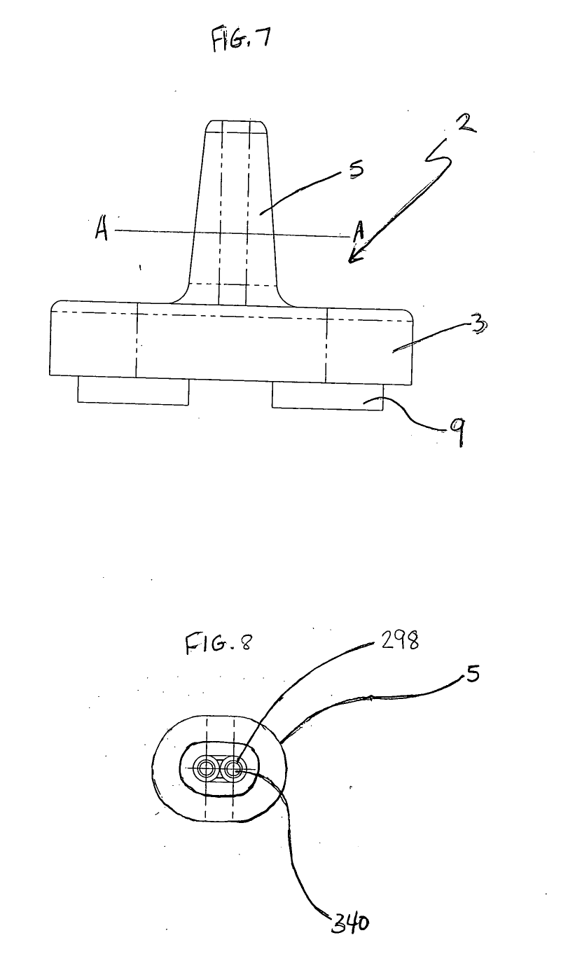

FIG. 12 is a side view of the connecting collar 12. The figure shows the relative positions of the upper connecting means 220 and the lower connecting means 200.

FIG. 13 is a longitudinal cross-section of the connecting collar 12 taken at line C-C of FIG. 12. The upper connecting means 220 is configured to have, in its top aspect, an annular depression into which flange 9 of the nasal spray head 2 fits during actuation of the device. The annular bayonet connection means 451 of the preferred embodiment has at its center a cylindrical opening through which the piston 320 passes. The person of skill will be aware that any annular connecting means which permits the piston to be positioned, as shown in FIG. 13, through an opening in the top of the connecting collar 12 is suitable for the device. The delivery means 250, at the uppermost portion of the upper end 270 is fitted with a corresponding connection means that mates with and connects to the bottom aspect of the annular bayonet-connection means 451. The lower connection means 200 is generally annular (either circular or any other geometric configuration in cross-section) in shape and is configured to allow the delivery means 250 to pass through its lower open end. Referring to FIG. 13, the lower connecting means 200 is shown with a threaded connection means 1301 for attachment of a correspondingly threaded liquid nasal composition container which, when connected to the lower connection means 200, surrounds and contains the lower portion of the delivery means 250 to allow the liquid nasal composition contained therein to be accessible for aspiration via the delivery means 250.

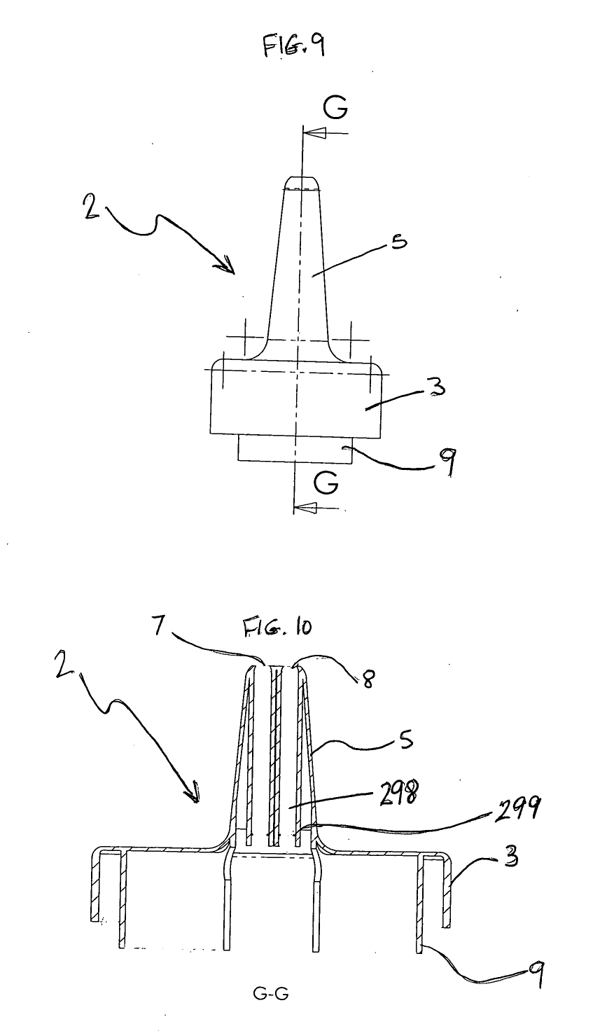

FIG. 14 illustrates an aspect of a preferred embodiment of the delivery means 250. Shown are the wider upper portions 260 and 270 of the delivery means 250, which accommodate the spring 300, the pump body 310 and the lower portion of the piston 320. FIG. 15 depicts a side view of the delivery means 250.

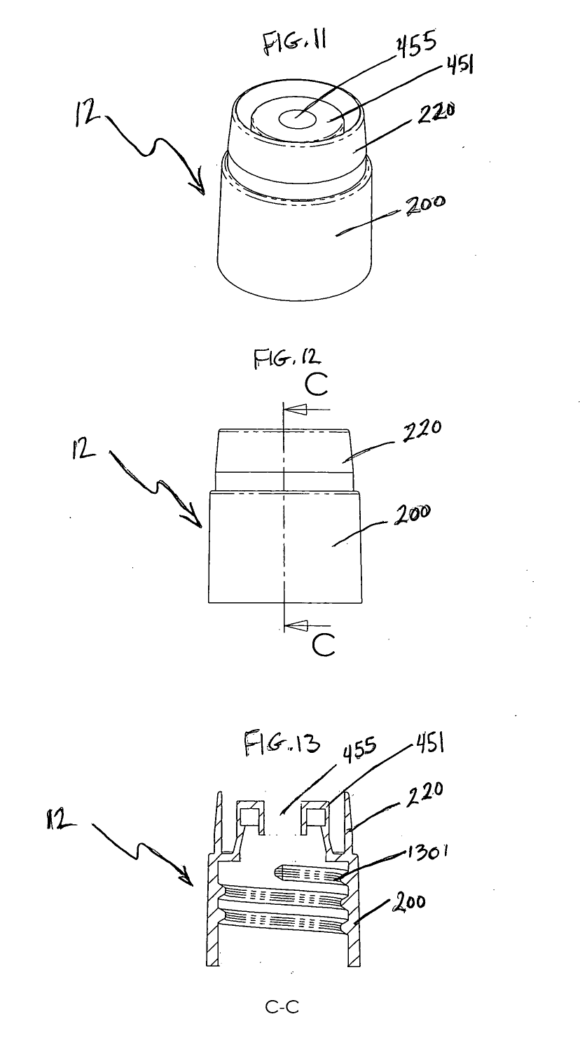

FIG. 16 is a longitudinal cross-section of the delivery means 250 taken at line F-F in FIG. 15. Referring to FIG. 16, at the upper end of the delivery means 250, is a connection means 450 designed to mate with and connect to the upper connecting means in the upper connecting means 220 of the connecting collar 12. The delivery means 250 comprises an open narrow lower portion 240 and a wider open upper portion 260, 270. The lower portion 240 of the delivery means 250 is inserted into the liquid nasal composition contained in the liquid nasal composition container for aspiration of the composition.

The upper portion 260 of the generally tubular delivery means 250, at its lower extreme, forms a perpendicular annular base 262 on which the lower end of the spring 300 rests. Between the upper extreme of the upper portion 260 and the lower extreme of the upper portion 260, the walls of the delivery means 250 form a second annular base 263, which functions as a stop for the piston 320 during actuation of the device. The annular stop 263 therefore preferably is configured in a diameter to permit passage of the pump body 310 and the lower portion of the piston 320 through the central space of the annulus, but with a diameter sufficiently narrow to prevent passage of the piston 320 beyond the piston stop 321.

In preferred embodiments, as shown in FIG. 16, the annular base 262 is a slot designed both to contain and support the spring 300 at the periphery and to form a raised, substantially horizontal central nest 261 on which the pump body 310 can rest. Preferably, the lower extreme 313 of the pump body 310 and the nest 261 are configured to mate such that the pump body 310 is stably maintained in its central position adjacent to and resting upon the nest 261 when the device is actuated and the spring 300 is compressed. When not in use, the spring 300 supports the pump body 310 above the nest 261. The lower portion of the pump body 310 thus is maintained in the interior of the spring 300 and in contact with the spring 300 on the lower face of its annular ridge 312 and is free to move downward upon actuation of the device.

FIG. 17 illustrates the pump body 310, including the annular ridge 312 and the lower extreme 313 thereof as described above. The pump body 310 is generally cylindrical in shape and is solid. At the upper end of the pump body 310 is a protrusion 314, preferably of a conical configuration. The protrusion 314 serves to seal the liquid transferring continuation 255 when the device is not actuated.

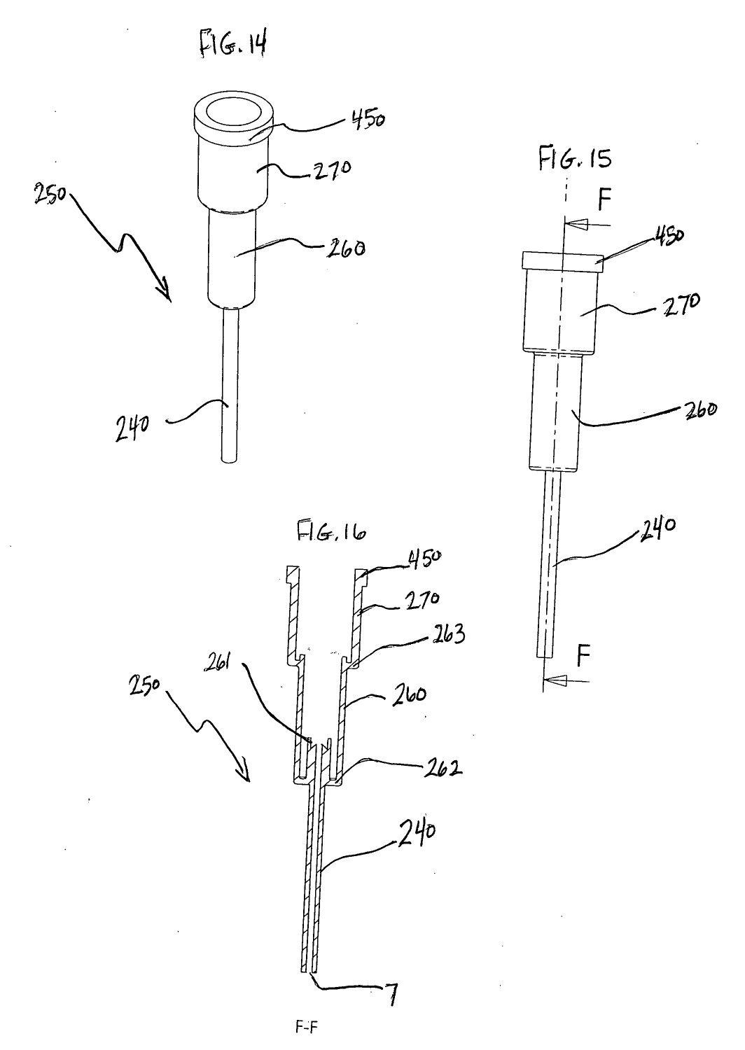

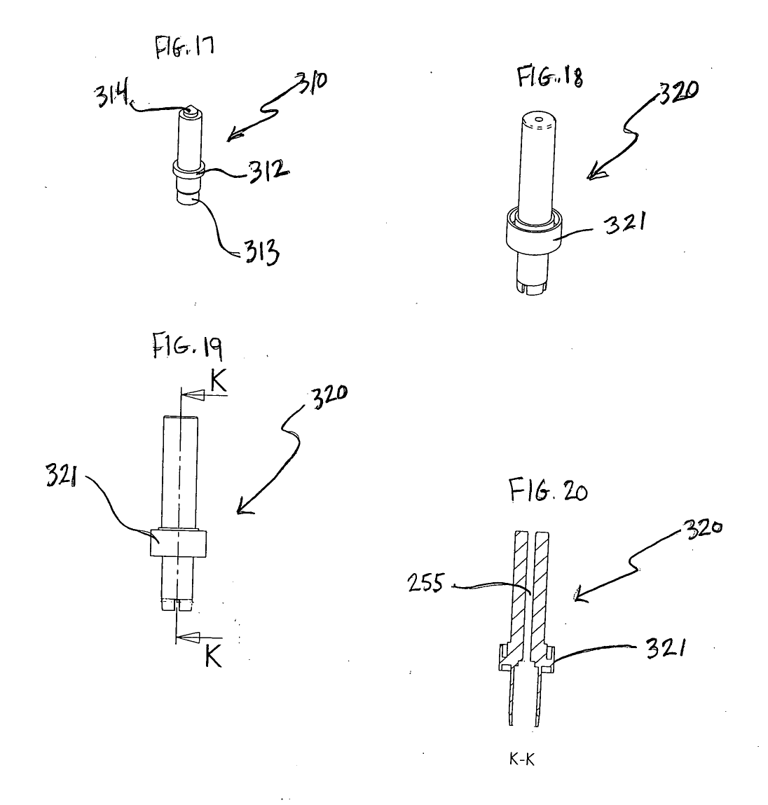

FIG. 18 shows the piston 320, including the piston stop 321 described above. The piston 320 is shown in side view in FIG. 19. FIG. 20 is a longitudinal cross-section view of the piston 320 taken at line K-K of FIG. 19. Referring to FIG. 20, the piston is a generally hollow, preferably cylindrical body as described above. The piston stop 321 is disposed on the exterior of the piston 320 between the upper and lower ends of the piston 320. The interior of the piston 320 defines a liquid accommodating channel having upper and lower ends.

The upper end of the pump body 310 is held in tension by the spring 300 in contact with the lower extreme of the liquid transferring continuation 255 until actuation of the nasal composition dispensing device. Upon actuation by pressing downward on the actuation means 9 of the nasal spray head 2, the pump body 310 is forced downward against the tension of the spring 300, which becomes compressed, by movement of the piston 320 inside the delivery conduit 250, opening the seal created by the protrusion 314 of the pump body 310 against the lower extreme of the liquid transferring continuation 255. Thus, the space formerly occupied by the pump body 310 prior to actuation and now above the pump body 310 fills with liquid nasal composition via the delivery conduit 250. Preferably, the amount of liquid nasal composition is metered by control of the volume of the space.

Release of the nasal spray head 2 by the user after actuation causes the upward movement of the pump body 310 in response to force delivered by the compressed spring 300. Liquid nasal composition in the space above the pump body 310 thus is forced upward through the liquid transferring continuation 255 in the interior of the piston 320.

FIGS. 21 and 22 illustrate the crossfeed 330 in exterior and bottom views, respectively. FIG. 23 is a longitudinal cross-section of the crossfeed 330 taken at line L-L of FIG. 22. The crossfeed 330 is an L-shaped hollow body that forms a further liquid transferring conduit 256, allowing liquid to be transferred from the upper end of the piston 320 and transversely or centrally. Thus, the liquid nasal compositions travels within the two liquid transferring conduits 256 in sufficient proximity to each other for dispensing through the openings 7, 8 in the nasal spray head 2 into the nostril of the user. The upper end of each crossfeed 330 fits in a liquid sealing manner into the lower end of the corresponding insert 340 (see FIG. 5).

FIGS. 24, 25 and 26 show exterior and two side views of the insert 340 taken at 90 degree angles to one another, respectively. FIG. 27 is a longitudinal cross-section view of the insert 340 taken at line M-M of FIG. 26. As discussed above, the lower portion of the insert 340 is a hollow body that defines a liquid transferring conduit 257 which is continuous with the liquid transferring conduit 256 of the crossfeed 330 and which exits at the exit port 288. The exit port 288 is located laterally to the liquid transferring conduit 257, such that the liquid nasal composition exits laterally, through the wall of the cylindrical body of the insert 340 into the cylindrical chambers 298 of the nasal spray head 2. Referring to FIGS. 25 and 26, the insert 340 optionally has a seal 342 below the level of the exit port 288. The upper portion of the insert 340, located above the exit port 288, is a solid, cylindrical body which is configured to fit inside the cylindrical chambers 298 of the nasal spray head 2.

It will be apparent to one of skill in the art that the dual nasal composition dispenser of the present invention as rendered in the figures discussed above may equally suitably be configured according to commonly used and well-known principles concerning actuatable nasal dispenser devices that are known in the art. Pump or spray containers as known in the art are suitable for dispensing of the liquid nasal compositions according to the present invention when configured to operate generally as shown in the accompanying figures and discussed above to dispense two separate liquid compositions which remain separate until each exits the device into the nostril of a user, through separate openings in a nasal insertion member.

The first and second nasal compositions preferably are delivered in metered doses in a micronized or aerosolized form. Each of the first and second nasal compositions may comprise any pharmaceutical composition suitable for nasal administration in a liquid vehicle. Any medication or other composition which can be administered nasally is contemplated for use with the present invention. Compounds which currently are administered only intravenously are especially advantageously used, to avoid the use of needles for injection and their concomitant dangers and discomfort. In addition, drugs which are commonly administered in combination with each other may be more conveniently administered together using the methods and devices of the present invention, for example common childhood vaccines that cannot be mixed together, such as polio vaccine and measles-mumps-rubella (MMR) vaccine.

Preferred nasal compositions are antihistamine drug compositions and nasal steroid drug compositions. However, any other compositions which are suitable or which can be formulated for nasal administration may be used with the device of the invention. For example, any of the known antihistamines, corticosteroids, decongestants and any pharmaceutically acceptable salts thereof, which are effective when applied topically to the nasal mucosa in an aqueous or other mucosally compatible solution, suspension or other topical preparation, may be used in the present invention. Any antihistamine or its pharmaceutically acceptable salts also may be used. Preferred antihistamines for use with the present invention include azelatine, cetirizine, desloratidine, fexofenadine or any pharmaceutically acceptable salt thereof. Using the present invention, any of these antihistamine compounds can be combined in treatment with any known steroid that is active when applied topically to the nasal mucosa, in spite of any incompatibilities of the compounds due to solubility differences, chemical instability, or the like.

Dosages of antihistamine for nasal application can be easily determined by the skilled clinician. The known antihistamine azelatine (Astelin®), which is administered nasally, serves as a guide for determining a suitable dose for any other antihistamines for topical nasal administration. Therefore, combination treatment for SAR, PAR, or other allergic rhinitis generally contains about 1 μg to about 10 mg, preferably about 10 μg to about 250 μg, and most preferably about 100 μg to about 150 μg (per dose) antihistamine compound. Clinicians generally have a great deal of experience with these types of well-known antihistamine compounds and can easily determine a suitable dose for use in combination with any of the known nasally active steroids. Appropriate doses for the nasally active steroid in the inventive combination medication can follow current FDA guidelines and are easily determined by the skilled clinician. Generally, compositions for use with the present invention contain about 1 μg to about 1 mg, preferably about 30 μg to about 80 μg, and most preferably about 45 μg to about 65 μg steroid compound per dose.

Any of the following antihistamine compounds, or any antihistamine compound, are contemplated for use with the present invention described here: acrivastine, azatadine, azelatine, brompheniramine, carbmoxamine, cetirizine, chlorpheniramine, clemastine, cyproheptadine, desloratidine, dexbrompheniramine, dexchlorpheniramine, diphenhydramine, doxylamine, fexofenadine, hydroxyzine, levocabastine, loratadine, meclizine, methdilazine, methscopolamine, phenindamine, pheniramine, phenylpropanolamine, promethazine, pyrilamine, rocastine, terfenidine, trimeprazine, tripelennamine, tripolidine, and triprolidine. Any of the following steroid compounds, or any corticosteroid compound, are contemplated for use with the present invention described here: beclomethasone, budesonide, dexamethasone, flunisolide, fluticasone, mometasone and triamcinolone.

TABLE I
Exemplary and Preferred Nasal Antihistamine/
Steroid Combination Treatments.

| Example | Antihistamine | Steroid |
|---|---|---|
| 1 | desloratidine | mometasone |
| 2 | loradidine | mometasone |
| 3 | fexofenadine | triamcinolone |
| 4 | cetirizine | fluticasone |
| 5 | azelatine | budesonide |

Additional groups of nasal compositions which may be used with the nasal device of the invention include, but are not limited to: insulin and an antihyperglycemic drug such as metformin or glyburide; long-acting insulin and short-acting insulin; any two or more vaccines; any two or more antiHIV drugs; or any desirable combination of drugs which are used concomitantly and can conveniently be administered nasally.

Additional compounds which are contemplated for nasal delivery with the present invention include but are not limited to: saline solution; sexual dysfunction compounds such as apomorphine hydrochloride, sildenafil, tadalafil and vardenafil; pain management compounds such as morphine gluconate, ketamine, codeine, buprenorphine and butorphanol; cytokines such as interferon beta, interferon alpha, interleukin-12; migraine medications such as sumatriptan, zolmitriptan and propranolol; hormones such as somatotropin, human growth hormone, estrogen, progesterone, testosterone and estradiol; osteoporosis medications such as alendronate, resedronate, calcitonin, raloxifene and teriparatide; insulins, including short acting insulin and long acting insulin; antihyperglycemia medications such as glyburide, biguanides (e.g., metformin), sulfonylureas (e.g., tolbutamide, chlorpropamide, glyburide, glipizide, glinepiride), meglitinides (e.g., repoglinide), alpha glucosidase inhibitors (e.g., acarbose, miglitol), thiazolidinediones (e.g., rosiglitazone, dioglitazone) and combination antihyperglycemia medications (e.g., glyburide/metformin and D-phenylalanine derivative/nateglinide); vitamins such as vitamin A, vitamin B12, vitamin B1, vitamin B2, vitamin B3, vitamin B5, vitamin B6, vitamin C, vitamin D and vitamin E; minerals such as zinc; nutraceuticals such as grapefruit seed extract, antioxidants, vitamins and probiotics; sedatives such as midazolam and other benzodiazepines, and fentanyl; motion sickness medications such as promethazine and scopolamine; Parkinson's disease medications such as levodopa and carbodopa; immunogens for induction of airway allegen tolerance; medications for stroke protection such as insulin-like growth factor I; gene therapy compounds such as viral vectors containing beneficial gene constructs; otitis media treatment compounds such as surfactants; treatments for cognition and neuronal survival such as glucagon-like peptide-1 antagonist and exendin; medications for real or suspected narcotic overdose such as naloxone; asthma medications such as albuterol; anticonvulsants such as phenobarbital, diazepam, lorazepam and the like; antiemetics such as metaclopramide; infertility treatment compounds such as leuprolide; growth factors such as somatotropin, nerve growth factor, and the like; antiviral drugs such as abacavir, amprenavir, cidovir, ddC, ddI, didanosine, efavirenz, indinavir, lamivudine, nelfinavir, nevirapine, retrovir, ritonavir, saquinavir, stavudine, tenofovir, zalcitabine, zidovudine and 3TC; antimicrobial drugs such as amebocides (e.g., paromomycin, yodoxin), antifungals (e.g., clotrimazole, fluconazole, ketoconazole, nystatin, griseofulvin, terbinafine, itraconazole, flucytosine, grispeg, clotrimazole, voriconazole, posaconazole, amphotericin B, caspofungin), antimalarials (e.g., chloroquine, hydroxychloroquine, mefloquine, pyrimethamine, pyrimethamine/sulfadoxine combination, halofantrine, primaquine), antiprotozoals (e.g., pentamidine, atovaquone), antimycobacterials (e.g., ethambutol, isoniazid, pyrazinamide, rifampin, rifabutin, rifapentine, rifampin/ isoniazid combination, rifampin/isoniazid/pyrazinamide combination, ethionamide); leprostatics such as dapsone and clofazimine; cardiac drugs such as nitroglycerine, epinephrine, lidocaine and propranolol; and vaccines against any disease or microorganism, for example tuberculosis, influenza, smallpox, rotavirus, salmonella, anthrax, meningocci, HIV, diphtheria, coxsackievirus, rhinovirus, plague, brucellosis, pertussis, adenoviruses, herpes viruses, streptococci, SARS, avian flu, yellow fever, recombinant raccoon virus, measles, mumps, rubella, polio, Epstein-Barr virus, para-influenza viruses, respiratory syncytial virus, cytomegalovirus, papillomaviruses, tetanus and ricin. Vaccines may be administered with any suitable adjuvant, such as Protollinm™. See Table II, below, for exemplary combinations of compounds which are advantageously used with the present invention. As will be appreciaed by those of skill in the art, combined medications may be provided in either or both nasal composition containers, allowing a dual dispensing device to dispense more than 2 compositions. See Table II.

TABLE II
Exemplary and Preferred Nasal Combination Treatments.

| Example | Compound(s) | Compound(s) |
|---|---|---|
| 6 | long-acting insulin | short-acting insulin |
| 7 | insulin | metformin/glyburide combination |
| 8 | polio vaccine | measles-mumps-rubella vaccine |
| 9 | smallpox vaccine | anthrax vaccine |
| 10 | promethazine | fluticasone |
| 11 | HIV vaccine | HCV vaccine |
| 12 | azelatine | saline solution |
| 13 | estrogen | progesterone |
| 14 | fat soluble vitamin | water soluble vitamin |
| | combination | combination |
| 15 | Lactobacillus plantarum | tetanus vaccine |
| | vaccine | |
| 16 | interferon alpha | interleukin-12 |
| 17 | lopinavir | ritonavir |
| 18 | epinephrine | lidocaine |
| 19 | nitroglycerin | propranolol |
| 20 | saline solution | fluticasone |
| 21 | promethazine/saline | triamcinolone |
| | solution | |

The foregoing has described the principles, embodiments, and modes of operation of the present invention. However, the invention should not be construed as being limited to the particular embodiments described above, as they should be regarded as being illustrative and not as restrictive. It should be appreciated that variations may be made in those embodiments by those skilled in the art without departing from the scope of the present invention.

Obviously, numerous modifications and variations he present invention are possible in light of the above teachings. It is therefore to be understood that the invention may be practiced otherwise than as specifically described herein.

What is claimed:

1. A dual nasal applicator system for simultaneously delivering a first nasal drug composition and a second nasal drug composition housed separately in first and second nasal composition chambers, respectively, comprising:
a spray head device comprising a nasal insertion portion having a body member and first and second openings extending through said body member, and an actuation portion that is integral with said nasal insertion portion for delivering the first and second nasal drug compositions through said first and second openings, respectively, when actuated;
a first connecting collar device having a first lower connecting portion and a first upper connecting portion, said first lower connecting portion being sealably attached to the first composition chamber and said first upper connecting portion being attached to said spray head device;
a second connecting collar device having a second lower connecting portion and a second upper connecting portion, said second lower connecting portion being sealably attached to the second composition chamber and said second upper connecting portion being attached to said spray head device;
a dual dispenser system having a first metered dispenser device and a second metered dispenser device;
said first dispenser device comprises a first lower portion member extending through and beneath said first connecting collar device to form a first liquid conduit from the first composition chamber, and a first upper portion member communicable with said first lower portion member for delivering a metered volume of the first nasal drug composition housed in the first composition chamber to said first opening of said spray head; and
said second dispenser device comprises a second lower portion member extending through and beneath said second connecting collar device to form a second liquid conduit from the second composition chamber, and a second upper portion member communicable with said second lower portion member for delivering a metered volume of the second nasal drug composition housed in the second composition chamber to said second opening of said spray head,
wherein the first and second nasal drug compositions remain separated until dispensed into a user's nares.

2. The system of claim 1, wherein said spray head device has at least one of an oblong shape, circular shape, square shape, rectangular shape, and octagonal shape, and is configured in different widths and heights.

3. The system of claim 1, wherein said first nasal composition and said second nasal composition are dispensed in equal volumes.

4. The system of claim 1, wherein said first nasal composition and said second nasal composition are dispensed in unequal volumes.

5. The system of claim 1, wherein said first nasal composition is an antihistamine drug composition and said second nasal composition is a nasal steroid drug composition.

6. The system of claim 5, wherein said antihistamine drug composition comprises an antihistamine drug selected from the group consisting of loratadine, desloratidine, terfenidine, fexofenadine, cetirizine, azelatine, azatadine, clemastine, brompheniramine, chlorpheniramine, dexbrompheniramine, diphenhydramine, doxylamine, phenindamine, pheniramine, pyrilamine, triprolidine, levocabastine, acrivastine, carbmoxamine, dexchlorpheniramine, promethazine, trimeprazine, methdilazine, hydroxyzine, rocastine, tripelennamine, meclizine, tripolidine, cyproheptadine, methscopolamine and phenylpropanolamine.

7. The system of claim 5, wherein said nasal steroid drug composition comprises a nasal steroid drug selected from the group consisting of fluticasone, dexamethasone, flunisolide, budesonide, triamcinolone and beclomethasone.
